(19) United States
(12) Patent Application Publication
(10) Pub. No.: US 2025/0387618 A1
(43) Pub. Date: Dec. 25, 2025

(54) NEURAL PROBE AND METHOD OF INTERFACING THE SAME WITH NEURONS IN VIVO

(71) Applicant: UNIST (ULSAN NATIONAL INSTITUTE OF SCIENCE AND TECHNOLOGY), Ulsan (KR)

(72) Inventor: Youngbin TCHOE, Ulsan (KR)

(73) Assignee: UNIST (ULSAN NATIONAL INSTITUTE OF SCIENCE AND TECHNOLOGY), Ulsan (KR)

(21) Appl. No.: 18/891,364

(22) Filed: Sep. 20, 2024

(30) Foreign Application Priority Data
Jun. 24, 2024 (KR) 10-2024-0082097

(51) Int. Cl. A61N 1/05; C12N 5/0793

(52) U.S. Cl. CPC A61N 1/0531; C12N 5/0619

(57) ABSTRACT

Provided are a neural probe and a method of interfacing the neural probe with neurons in vivo. In one embodiment, the neural probe may include a main body, holes, interface neurons disposed in each hole, and intracellular recording interfaces disposed in each hole, configured to measure the intracellular potentials of the interface neurons. The interface neurons on the neural probe may interconnect with the neurons in the intact brain.

### CROSS-REFERENCE TO RELATED APPLICATION

This application claims the benefit of Korean Patent Application No. 10-2024-0082097, filed on Jun. 24, 2024, in the Korean Intellectual Property Office, the disclosure of which is incorporated herein in its entirety by reference.

### BACKGROUND

### 1. Field

The disclosure relates to a neural probe.

### [Acknowledgements]

This research was supported by the Samsung Future Technology Promotion Project (Project No.: SRFC-IT2302-02).

### 2. Description of the Related Art

The transmission of a nerve signal (i.e., an electrical signal) is based on a change in a membrane potential. When a neuron in a resting state receives an external stimulus, ion channels of a cell membrane open, causing an increase in membrane potential. An external stimulus greater than a threshold generates an action potential that depolarizes the neuron. When the action potential arrives at an axon terminal through an axon, a neurotransmitter is emitted through a synapse. The neurotransmitter operates as an external stimulus to the next neuron, and through this process, a nerve signal is transmitted between neurons.

Accurately measuring nerve signals is important in various fields, such as brain-computer interfaces, neuroscience, neurological disease research, and the like. Conventional neural probes have relied on extracellular measurement of neural activities, which suffers from a low signal-to-noise ratio. For accurate measurement of neural activities, it is crucial to perform intracellular recording. To this end, a neural probe capable of effectively interfacing with neurons intracellulay in vivo is required.

### SUMMARY

Provided are a neuron-integrated neural probe and a method of interfacing the neural probe with neurons intracellularly in vivo

Provided are a neuron-integrated neural probe and a method of interfacing the neural probe with neurons intracellularly in vivo.

According to an aspect of the disclosure, provided is a neural probe for interfacing with neurons in vivo.

The neural probe according to an embodiment may include a main body, a hole including a first opening formed in an upper surface of the main body and a second opening formed in a lower surface of the main body, and an intracellular recording interface disposed in the hole and configured to measure an intracellular potential.

The neural probe may further include an interface neuron disposed in the hole.

The interface neuron may form a synaptic connection in vivo to a neuron in a body.

The neural probe may further include a culture medium disposed in the hole to allow the interface neuron to grow beyond at least one of the first opening and the second opening.

The neural probe may further include a chemical substance disposed in the hole to promote growth of the interface neuron.

The intracellular recording interface may be configured to measure an intracellular potential of the interface neuron.

The intracellular recording interface may be located at the same distance from the first opening and the second opening.

The neural probe may further include an interface neuron disposed in the hole, wherein the intracellular recording interface may include a protruding electrode configured to measure the intracellular potential of the interface neuron.

The protruding electrode may extend from an inner surface of the hole to an inner space of the hole.

The protruding electrode may extend in a direction intersecting a height direction of the main body.

The protruding electrode may penetrate a cell membrane of the interface neuron.

The neural probe may further include an insulating film covering a portion of the protruding electrode exposed to outside of the interface neuron.

The protruding electrode may be configured to apply an electrical stimulus to the interface neuron to promote growth of the interface neuron.

The neural probe may further include an interface neuron disposed in the hole, wherein the intracellular recording interface may include a planar patch clamp configured to measure an intracellular potential of the interface neuron.

The first opening and the second opening may have the same size.

The main body may include an upper main body in which the first opening is formed and a lower main body in which the second opening is formed.

The neural probe may further include a signal transmission path through which an intracellular potential measured by the intracellular recording interface is transmitted to outside.

The neural probe may further include a first membrane covering the first opening.

The neural probe may further include a second membrane covering the second opening.

According to another aspect of the disclosure, provided is a method of interfacing a neural probe with neurons in vivo.

The method of interfacing a neural probe with neurons in vivo may include implanting a neural probe in a body, growing an interface neuron disposed in a hole of a neural probe, creating a synaptic connection between the interface neuron and a neuron in the body, and measuring an intracellular potential of the interface neuron by using an intracellular recording interface of the neural probe.

### BRIEF DESCRIPTION OF THE DRAWINGS

These and/or other aspects will become apparent and more readily appreciated from the following description of the embodiments, taken in conjunction with the accompanying drawings in which:

FIGS. 1A to 1C are views for describing a neural probe according to an embodiment;

FIG. 2A is a view for describing an intracellular recording interface according to an embodiment;

FIG. 2B is a view for describing an interface neuron according to an embodiment;

FIG. 2C is a view for describing membranes according to an embodiment;

FIG. 2D is a view for describing a connection between neurons in the body and an interface neuron according to an embodiment;

FIG. 3 is a view for describing interfacing between the neurons in the body and a neural probe according to an embodiment;

FIG. 4A is a view for describing a neural probe according to an embodiment;

FIG. 4B is an enlarged view of a hole of a neural probe according to an embodiment;

FIGS. 5 and 6 are enlarged views of a hole of a neural probe according to some embodiments;

FIG. 7 is a graph showing an anticipated intracellular potential according to an embodiment;

FIG. 8 is a view for describing a neural probe according to an embodiment;

FIGS. 9A and 9B are enlarged views of a recess of a neural probe according to some embodiments;

FIG. 10 is a view for describing a neural probe according to an embodiment;

FIGS. 11A and 11B are enlarged views of a recess of a neural probe according to some embodiments;

FIGS. 12A and 12B are enlarged views of a recess of a neural probe according to some embodiments;

FIGS. 13A and 13B are view for describing a neural chip according to some embodiments; and

FIG. 14 is a flowchart showing a method of interfacing a neural probe with neurons in vivo, according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

In the disclosure, expressions such as “at least one of a, b, or c” may denote “a”, “b”, “c”, “a and b”, “a and c”, “b and c”, “all of a, b, and c”, or modifications thereof.

The terms used in the embodiments have been selected from currently widely used general terms in consideration of the functions in the disclosure. However, the terms may vary according to the intention of one of ordinary skill in the art, case precedents, and the advent of new technologies. Furthermore, for special cases, meanings of the terms selected by the applicant are described in detail in the description section. Accordingly, the terms used in the disclosure are defined based on their meanings in relation to the contents discussed throughout the specification, not by their simple meanings.

As used herein, the singular forms are intended to include the plural forms as well, unless the context clearly indicates otherwise. Unless defined otherwise, all terms used herein including technical or scientific terms have the same meanings as those generally understood by those of ordinary skill in the art to which the disclosure may pertain. Furthermore, in the disclosure, terms such as “first” and “second” are used herein merely to describe a variety of components, but the components are not limited by the terms. The terms are used only for the purpose of distinguishing one component from another component.

Throughout the disclosure, when a portion “comprises” a component, unless stated otherwise, the presence or addition of one or more other features or components is not precluded.

Hereinafter, with reference to the accompanying drawings, the embodiment of the present invention will be described in detail so that a person skill in the art to which the present invention pertains can easily implement the invention. However, the disclosure may be implemented in a variety of different forms, but not limited to the embodiments described herein.

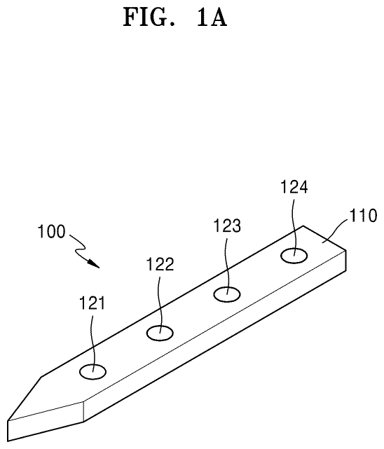

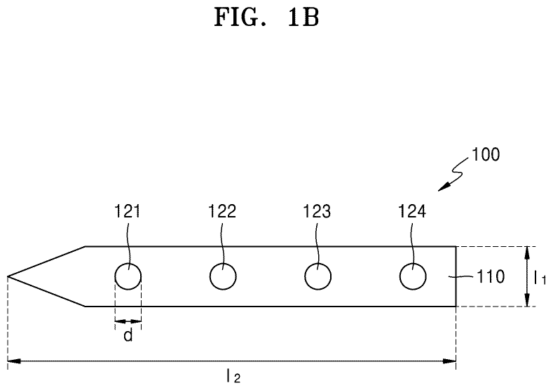

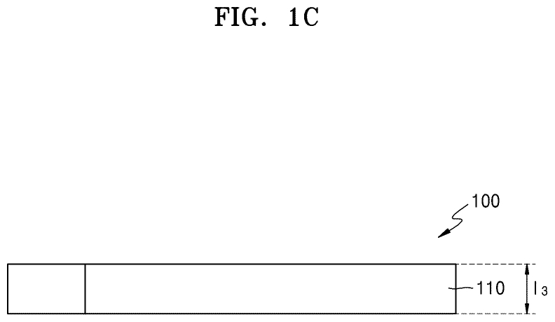

FIGS. 1A to 1C are views for describing a neural probe 100 according to an embodiment.

The neural probe 100 may be a device to interface with neurons in a body. To this end, the neural probe 100 may be implanted in the body. In detail, the neural probe 100 may be implanted in the body to interact with a central nervous system (CNS) or a peripheral nervous system (PNS).

In an embodiment, the neural probe 100 may be implanted in a brain for a brain computer interface (BCI), brain surgery, neural prosthetics, brain-to-brain communication or the like. For example, in a case of a brain-computer interface, the neural probe 100 may be used to manipulate a computer via thoughts. For example, in a case of brain surgery, the neural probe 100 may be used to find lesion tissues in brain tumor or epilepsy patients through precise electrical measurements.

The neural probe 100 may measure nerve signals (i.e., electrical signals) of neurons in the body. The neural probe 100 may include interface neurons. When the neuron-integrated neural probe 100 is implanted in the body, the interface neurons may interface with the neurons intracellularly in vivo. When the neuron-integrated neural probe 100 is implanted in the body, the interface neurons may have synaptic connections with the neurons in the body. The neural probe 100 may measure the nerve signals of the neurons in the body by measuring the intracellular potentials of interface neurons.

In an embodiment, an interface neuron may be genetically the same neuron as a neuron in the body. Alternatively, when the interface neuron is genetically different from the neuron in the body, an immune inhibitor may be used. Furthermore, the interface neuron may be a neuron in an early stages of culture that has potency to rapidly extend its neurites.

The neural probe 100 may transmit electrical signals to the neurons in the body. The neural probe 100 may transmit electrical signals to the neurons in the body connected to the interface neurons by applying the electrical signals to the interface neurons. Furthermore, the neural probe 100 may stimulate the neurons in the body connected to the interface neurons by applying electrical signals to the interface neurons.

Referring to FIGS. 1A to 1C, in an embodiment, the neural probe 100 may include a main body 110, holes 121 to 124, and intracellular recording interfaces disposed in the holes 121 to 124. Although not illustrated in FIGS. 1A to 1C, the neural probe 100 may further include a connector for connection with an external device.

The main body 110 may include the holes 121 to 124. The main body 110 may include one or more holes, and the number of holes of the main body 110 is not limited by the embodiments with reference to FIGS. 1A to 1C.

The holes 121 to 124 may each include a first opening formed in an upper surface of the main body 110 and a second opening formed in a lower surface of the main body 110. In another embodiment, the first opening may be formed one surface of the main body 110, and the second opening may be formed in another surface of the main body 110. The locations of the first and second openings are not limited by the embodiments with reference to FIGS. 1A to 1C. In another embodiment, each of the holes 121 to 124 may have two or more openings, and the number of openings is not limited by the embodiments with reference to FIGS. 1A to 1C.

The intracellular recording interface configured to measure an intracellular potential may be disposed in each of the holes 121 to 124. Furthermore, the interface neuron may be disposed in each of the holes 121 to 124. The intracellular recording interface may be configured to measure the intracellular potential of the interface neuron. Furthermore, the intracellular recording interface may be configured to apply an electrical signal to the interface neuron.

As the intracellular recording interface and the interface neuron are disposed in each of holes 121 to 124, in an implantation process of the neural probe 100, damage to the intracellular recording interface and the interface neuron may be reduced.

The holes 121 to 124 may be disposed in various ways. For example, the holes 121 to 124 may be arranged side by side, zigzag, uniformly, or randomly in the longitudinal direction of the main body 110, but the disclosure is not limited thereto.

In an embodiment, the holes 121 to 124 may be uniformly arranged. The interface neurons may be uniformly arranged along the holes 121 to 124. The neural probe 100 may measure the nerve signals of the neurons in the body at high yield by using the interface neurons that are uniformly arranged.

In an embodiment, the holes 121 to 124 may be arranged at intervals suitable for connecting the interface neurons one to one to the neurons in the body. For example, when an interval between the neurons in the body is about 50 μm to 200 μm, the holes 121 to 124 may be arranged at intervals of about 50 μm to 200 μm. Accordingly, the possibility that one interface neuron is connected to a plurality of neurons in the body or a plurality of interface neurons are connected to one neuron in the body may be reduced.

The holes 121 to 124 may have diameters suitable for accommodating the interface neurons. For example, a diameter d of the hole 121 may be about 5 μm to about 200 μm, but the disclosure is not limited thereto.

The holes 121 to 124 may have various shapes. For example, the first and second openings of the hole 121 may have various shapes including a circle. For example, the first and second openings of the hole 121 may have the same size and shape or different sizes and shapes.

The main body 110 may include a material suitable for implantation within the body. For example, the main body 110 may include a flexible material. For example, the main body 110 may include at least one of parylene, polyimide, SU-8, Si, or silicon dioxide (SiO₂), but the disclosure is not limited thereto.

The main body 110 may have a shape suitable for implantation within the body. As illustrated in FIGS. 1A to 1C, the main body 110 may include a sharp end portion and have a shape extending in the longitudinal direction. The shape of the main body 110 is not limited to FIGS. 1A to 1C, and may have various shapes suitable for implantation within the body various shape.

The main body 110 may have a height suitable for accommodating interface neurons and for implantation within the body. For example, a height 13 of the main body 110 may be about 5 μm to about 200 μm, but the disclosure is not limited thereto.

The main body 110 may have a width 11 and a longitudinal length 12 suitable for accommodating a plurality of interface neurons and for implantation within the body. For example, the width 11 may be about 10 μm to about 200 μm, and the longitudinal length 12 may be about 1 mm to about 100 mm, but the disclosure is not limited thereto.

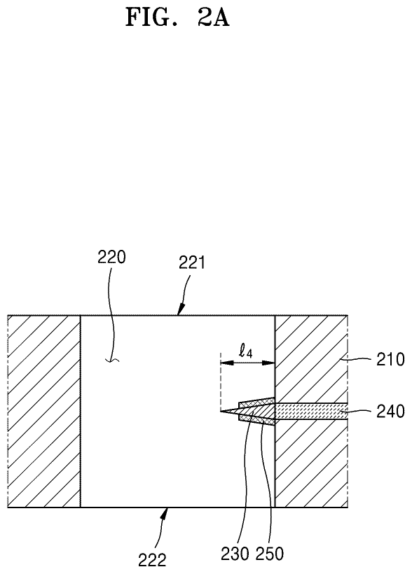

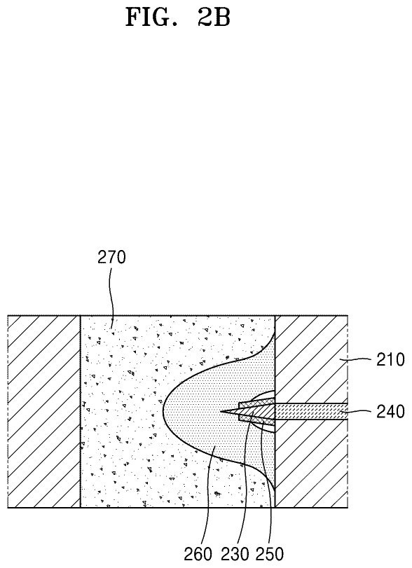

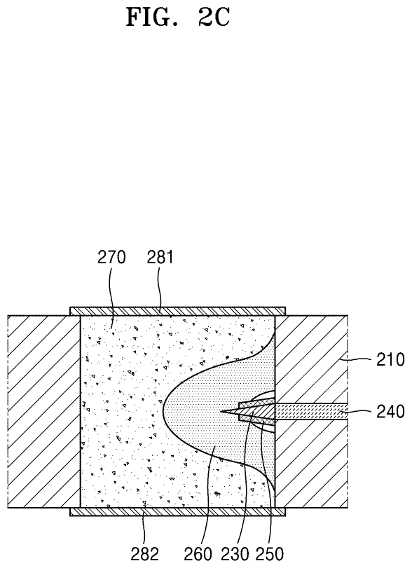

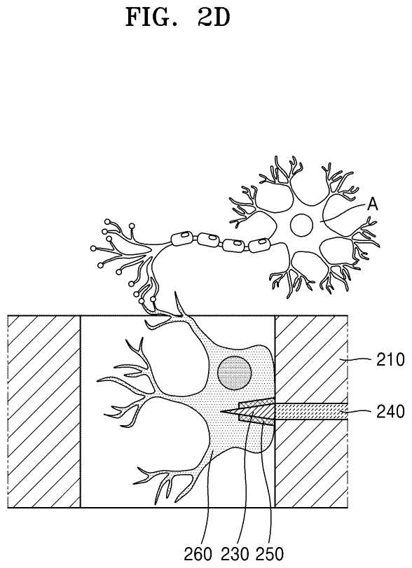

FIG. 2A is a view for describing an intracellular recording interface 230 according to an embodiment. FIG. 2B is a view for describing an interface neuron 260 according to an embodiment. FIG. 2C is a view for describing membranes 281 and 282 according to an embodiment.

FIG. 2A to 2C are enlarged views of a hole 220 formed in a main body 210 according to some embodiments.

Referring to FIG. 2A to 2C, the intracellular recording interface 230 and the interface neuron 260 may be disposed in the hole 220. In an embodiment, one interface neuron 260 may correspond to one intracellular recording interface 230. As one interface neuron 260 is arranged in one intracellular recording interface 230, a delicate neural probe capable of measuring the intracellular potential of the interface neuron 260 may be provided.

Unlike the illustrations of FIG. 2A to 2C, a plurality of intracellular recording interfaces and/or a plurality of interface neurons may be disposed in the hole 220. For example, one interface neuron may correspond to a plurality of intracellular recording interfaces. In this case, as the intracellular potential of one interface neuron is measured using a plurality of intracellular recording interfaces, a highly reliable measurement value may be obtained.

The intracellular recording interface 230 may be located at the same distance form a first opening 221 and a second opening 222. In other words, the intracellular recording interface 230 may be located in the middle area of the hole 220. Accordingly, the interface neuron 260 disposed in the intracellular recording interface 230 may also be located in the middle area of the hole 220. As a symmetrical structure is formed through such an arrangement, the interface neuron 260 may be connected to the nearest neuron in the body in any direction.

In an embodiment, the intracellular recording interface 230 may be a protruding electrode 230. The protruding electrode 230 may be configured to measure the intracellular potential of the interface neuron 260 may be configured to measure. To this end, the protruding electrode 230 may include a biocompatible conductive material, such as platinum, gold, silicon, iridium oxide (IrOx), or poly (3,4-ethylenedioxythiophene) polystyrene sulfonate (PEDOT:PSS), and the like.

The protruding electrode 230 may have various shapes. For example, the protruding electrode 230 may include one of a nanowire, a nanorod, and a nanotube but the disclosure is not limited thereto.

The protruding electrode 230 may extend from an inner surface of the hole 220 toward an inner space of the hole 220. The protruding electrode 230 may extend in a direction intersecting the height direction of the main body 210. For example, the first opening 221 and the second opening 222 may be arranged parallel to each other, and the protruding electrode 230 may extend parallel to a plane on which the first opening 221 is disposed. For example, the protruding electrode 230 may extend in the width direction or longitudinal direction of the main body 210.

The protruding electrode 230 may have a size suitable for measuring the intracellular potential of the interface neuron 260 by penetrating a cell membrane of the interface neuron 260. For example, a length 14 of the protruding electrode 230 may be about 1 μm to about 20 μm, and the diameter of a tip of the protruding electrode 230 may be about 1 nm to about 2 μm, but the disclosure is not limited thereto.

A portion other than the tip of the protruding electrode 230 may be covered by an insulating film 250. As a portion of the protruding electrode 230 exposed to the outside of the interface neuron 260 is covered by the insulating film 250, the accuracy and reliability in the measurement of an intracellular potential may be improved. In another embodiment, the insulating film 250 may be replaced with another type of membrane.

A culture medium 270 for growth of the interface neuron 260 may be disposed in the hole 220. In an embodiment, as alginic acid is added to the culture medium 270, hydrogel may be formed.

A chemical substance to promote the growth of the interface neuron 260 may be added to the culture medium 270 or hydrogel. For example, the chemical substance may include a brain-derived neurotrophic factor (BDNF), but the disclosure is not limited thereto.

A neural probe may promote the growth of the interface neuron 260 by applying an electrical stimulus to the interface neuron 260 through the protruding electrode 230. For example, the growth of the interface neuron 260 may be promoted by applying a voltage of a predetermined magnitude to the interface neuron 260 for a predetermined time through the protruding electrode 230.

In an embodiment, the neural probe may include at least any one of a first membrane 281 covering the first opening 221 or a second membrane 282 covering the second opening 222. The first and second membranes 281 and 282 may prevent the culture medium 270 from being exposed to air for a time during which the interface neuron 260 is cultured outside the body and implanted in the body. Furthermore, the first and second membranes 281 and 282 may prevent the interface neuron 260 or the culture medium 270 from escaping to the outside of the hole 220 in a neural probe implant process. The first and second membranes 281 and 282 may be removed by being biodegraded after the neural probe is implanted in the body.

The neural probe may include a signal transmission path 240 to transmit the intracellular potential measured by the intracellular recording interface 230 to the outside. In an embodiment, the intracellular potential measured by the intracellular recording interface 230 may be transmitted to an external device through the signal transmission path 240. For example, the signal transmission path 240 may be a wire, but the disclosure is not limited thereto.

FIG. 2D is a view for describing a connection between a neuron A in the body and the interface neuron 260, according to an embodiment.

When a neural probe is implanted in the body, neurites extending from the interface neuron 260 may be synaptically connected to the neuron A in the body. For example, a dendrite (or an axon) of the interface neuron 260 may be synaptically connected to an axon (or a dendrite) of the neuron A in the body. In an embodiment, the interface neuron 260 may receive a nerve signal of the neuron A in the body through a synaptic connection. The intracellular recording interface 230 may measure a nerve signal of the neuron A in the body by measuring the intracellular potential of the interface neuron 260. In an embodiment, an electrical signal may be applied to the interface neuron 260 through the intracellular recording interface 230. The electrical signal may be transmitted to the neuron A in the body synaptically connected to the interface neuron 260.

As the interface neuron 260 extends a neurite to create a synaptic connection (i.e., form a synaptic connection) to the neuron A in the body, a nerve signal of the neuron A in the body that is relatively far from the neural probe may be measured. Furthermore, as the interface neuron 260 grows according to the environment in the body, a neural probe that is adaptive to the environment in the body may be provided.

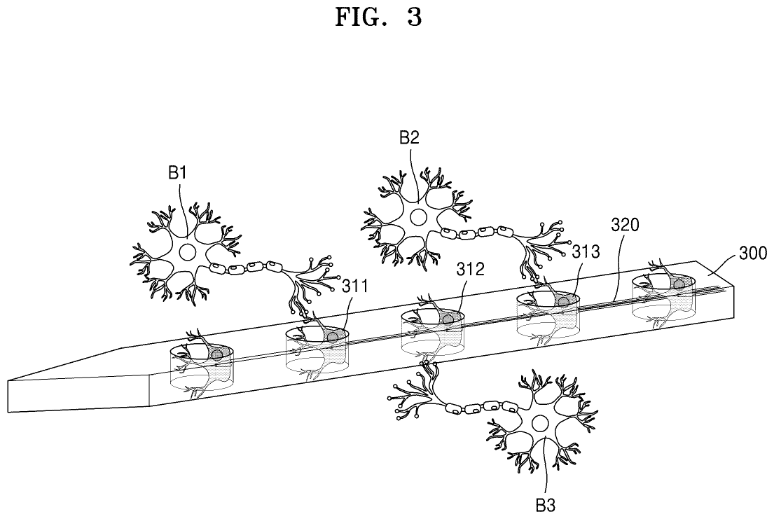

FIG. 3 is a view for describing interfacing between neurons B1, B2, and B3 in the body and a neural probe 300, according to an embodiment.

When the neural probe 300 is implanted in the body, an interface neuron 311 may grow to extend a neurite of the interface neuron 311 to the outside of a hole. The neurite of the interface neuron 311 may freely grow through a first opening or a second opening of the hole. Accordingly, the interface neuron 311 may create a synaptic connection to the nearest neuron in either direction.

In an embodiment, the first and third interface neurons 311 and 313 create a synaptic connection to the first and third neurons B1 and B3 located above an upper surface of the neural probe 300, and a second interface neuron 312 creates a synaptic connection to the second neuron B2 located under a lower surface of the neural probe 300. The intracellular potentials of the first to third interface neurons 311 to 313 measured by intracellular recording interfaces may be transmitted to an external device through a signal transmission path 320.

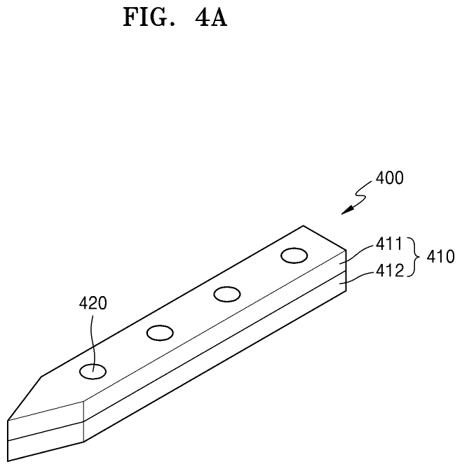

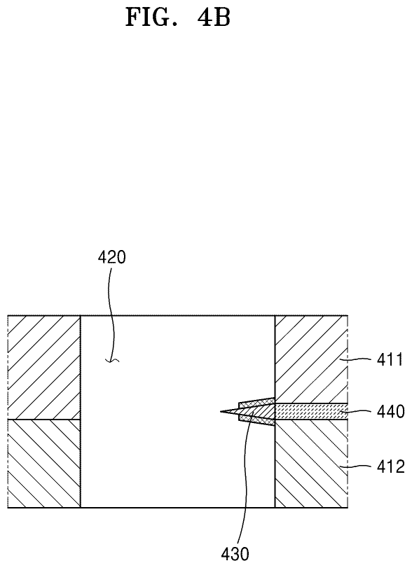

FIG. 4A is a view for describing the neural probe 400 according to an embodiment. FIG. 4B is an enlarged view of a hole 420 of the neural probe 400 according to an embodiment.

Referring to FIG. 4A and 4B, a main body 410 of the neural probe 400 may include an upper main body 411 and a lower main body 412. Such a configuration may facilitate the manufacture of the neural probe 400. For example, after the lower main body 412 is first disposed, an intracellular recording interface 430 and a signal transmission path 440 may be mounted on the lower main body 412. Then, by mounting the upper main body 411 on the lower main body 412 and the signal transmission path 440, the neural probe 400 may be easily assembled.

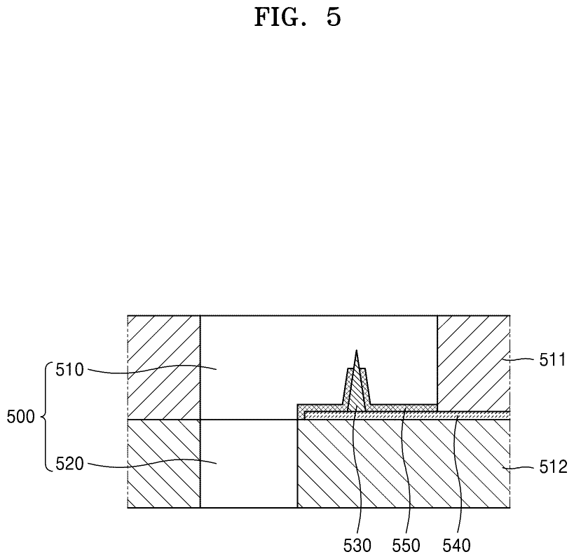

FIG. 5 is an enlarged view of a hole 500 of a neural probe according to an embodiment.

The hole 500 may include an upper hole 510 and a lower hole 520. The upper hole 510 may be formed in an upper main body 511, and the lower hole 520 may be formed in a lower main body 512. The upper hole 510 may have a diameter greater than a diameter of the lower hole 520. An intracellular recording interface 530 may be disposed on a step formed due to a difference in diameter between the upper hole 510 and the lower hole 520.

In an embodiment, the intracellular recording interface 530 may be a protruding electrode 530. The protruding electrode 530 may extend in a longitudinal direction (i.e., a height direction of a neural probe) of the hole 500. A portion other than the tip of the protruding electrode 530 and a portion of a signal transmission path 540 exposed to the inside of the hole 500 may be covered by an insulating film 550. The intracellular potential of an interface neuron measured by the protruding electrode 530 may be transmitted to an external device through the signal transmission path 540.

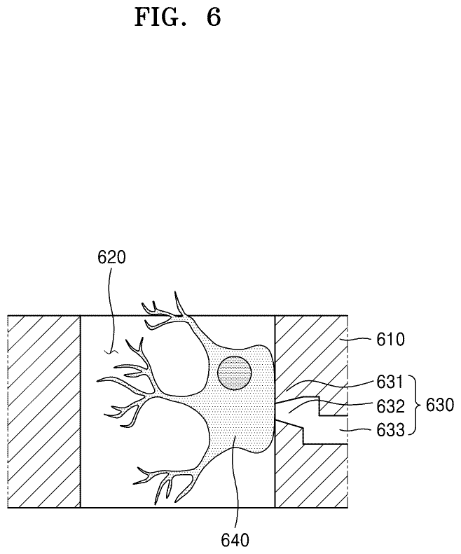

FIG. 6 is an enlarged view of a hole 620 of a neural probe according to an embodiment.

In an embodiment, the neural probe may include a planar patch clamp 630 as an intracellular recording interface. A main body 610 may form a planar substrate 631 of a planar patch clamp 630. The planar substrate 631 may enable an interface neuron 640 to be easily attached thereto and may have low capacitance properties. A micron-sized hole 632 of the planar patch clamp 630 may be formed in the main body 610. The interface neuron 640 may be disposed in the micron-sized hole 632. Furthermore, an intracellular bath 633 of the planar patch clamp 630 may be formed in the main body 610. The intracellular bath 633, as a signal transmission path, may perform a function of transmitting the intracellular potential of the interface neuron 640 measured by the planar patch clamp 630.

As the planar patch clamp 630 functions as an automated patch clamp, even after the neural probe is implanted in the body, the intracellular potential of the interface neuron 640 may be automatically measured without a manual operation. Furthermore, by using the planar patch clamp 630 as the intracellular recording interface, the intracellular potential of the interface neuron 640 may be measured with high accuracy.

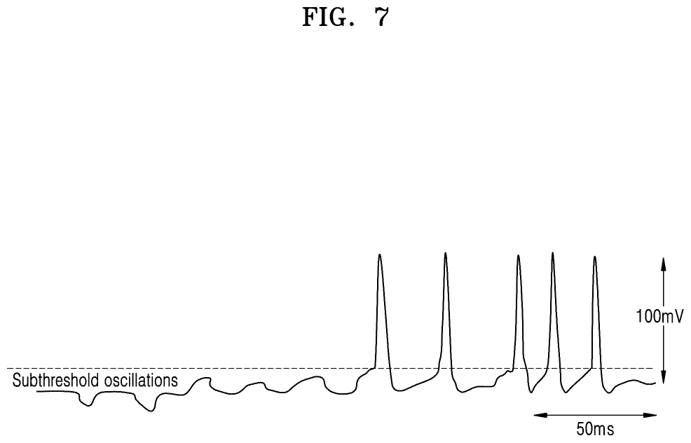

FIG. 7 is a graph showing an anticipated intracellular potential according to an embodiment.

A neural probe may include an intracellular recording interface and an interface neuron. The interface neuron creates a synaptic connection to a neuron in the body, and the intracellular recording interface measures the intracellular potential of the interface neuron. Accordingly, the neural probe may measure the intracellular potential of the interface neuron connected to the neuron in the body. The neural probe may measure an action potential of the interface neuron and a potential below a threshold (i.e., subthreshold oscillations) with a high signal-to-noise ratio (SNR) by measuring the intracellular potential. Furthermore, the neural probe may measure a nerve signal of a neuron in the body with a high SNR through the interface neuron.

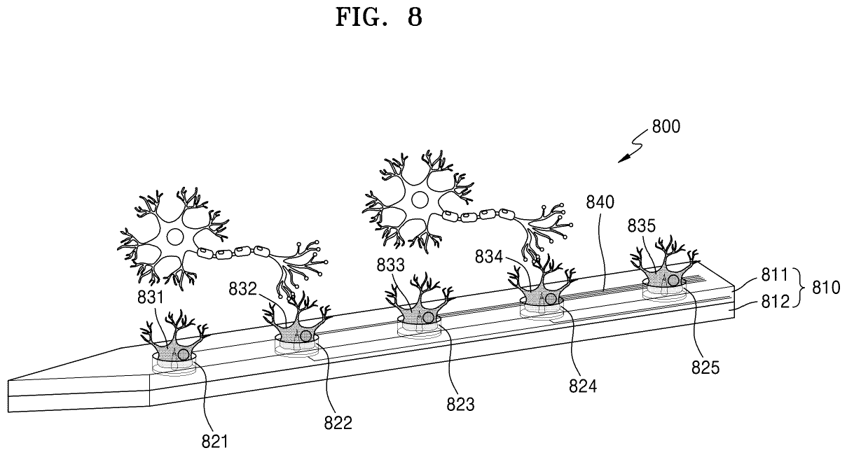

FIG. 8 is a view for describing a neural probe 800 according to an embodiment.

The descriptions according to some embodiments of the disclosure may be applied to the neural probe 800 of FIG. 8. Redundant descriptions thereof are omitted.

The neural probe 800 may include a main body 810, recesses 821 to 825, intracellular recording interfaces disposed in the recesses 821 to 825, and signal transmission paths 840. Although it is not illustrated in FIG. 8, the neural probe 800 may further include a connector for connection to an external device.

The main body 810 may include an upper main body 811 and a lower main body 812. The recesses 821 to 825 may be formed in the upper main body 811, and openings of the recesses 821 to 825 may be located in an upper surface of the upper main body 811. The main body 810 may include one or more recesses, and the number of recesses included in the main body 810 is not limited by the embodiment with reference to FIG. 8.

The intracellular recording interface configured to measure an intracellular potential may be disposed in each of the recesses 821 to 825. Interface neurons 831 to 835 may be disposed in the recesses 821 to 825, and the intracellular recording interface may be configured to measure the intracellular potential of an interface neuron.

The recesses 821 to 825 may be disposed in various ways. For example, the recesses 821 to 825 may be arranged side by side, zigzag, uniformly, or randomly in the longitudinal direction of the main body 810, but the disclosure is not limited thereto.

In an embodiment, the recesses 821 to 825 may be uniformly arranged. The interface neurons 831 to 835 may be uniformly arranged along the recesses 821 to 825. The neural probe 800 may measure nerve signals of neurons in the body at a high yield by using the interface neurons 831 to 835 that are uniformly arranged.

In an embodiment, the recesses 821 to 825 may be arranged at intervals suitable for connecting the interface neurons 831 to 835 one to one to the neurons in the body. Accordingly, the possibility that one interface neuron is connected to a plurality of neurons in the body or a plurality of interface neurons are connected to one neuron in the body may be reduced.

The recesses 821 to 825 may each have a diameter suitable for accommodating the interface neurons 831 to 835. For example, the diameter of the recess 821 may be about 5 μm to about 200 μm, but the disclosure is not limited thereto.

The recesses 821 to 825 may have various shapes. For example, an opening of the recess 821 may have various shapes including a circle.

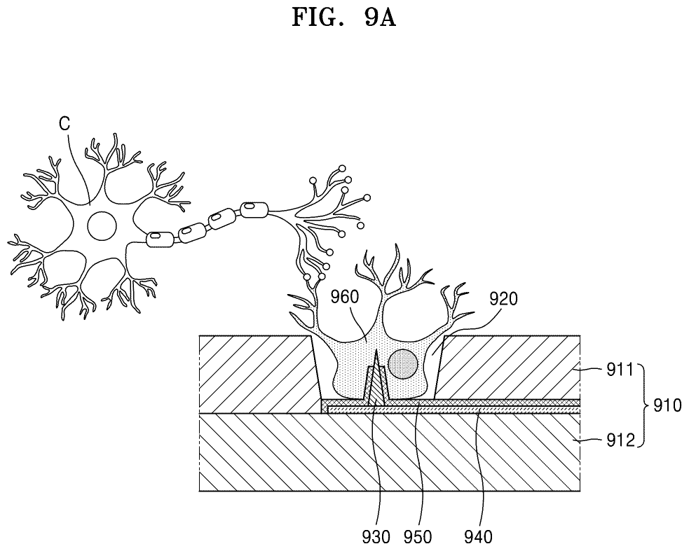

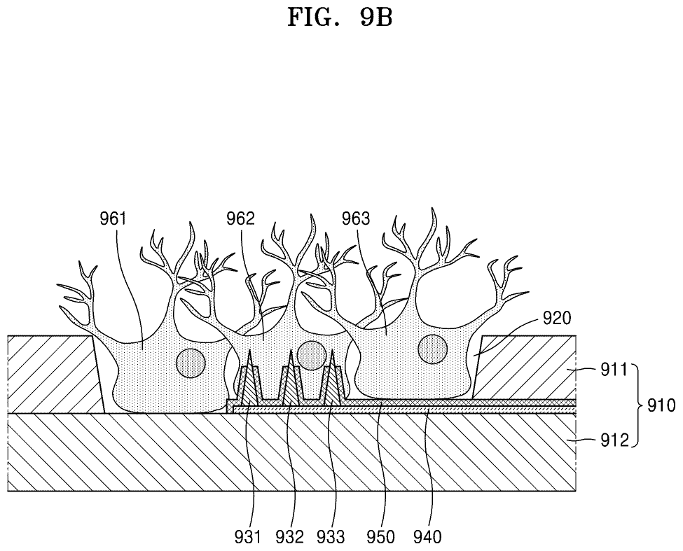

FIGS. 9A and 9B are enlarged views of a recess 920 of a neural probe according to some embodiments.

The descriptions according to some embodiments of the disclosure may be applied to the neural probe of FIGS. 9A and 9B. Redundant descriptions thereof are omitted.

Referring to FIG. 9A, a recess 920 may be formed in an upper main body 911. An opening of the recess 920 may be located in an upper surface of the upper main body 911.

An intracellular recording interface 930 may be a protruding electrode 930. The protruding electrode 930 may extend from an upper surface of a lower main body 912 toward an inner space of the recess 920. For example, the protruding electrode 230 may extend in a height direction of a main body 910.

A portion other than the tip of the protruding electrode 930 and a portion of a signal transmission path 940 exposed to the inside of the recess 920 may be covered by an insulating film 950.

Although it is not illustrated in FIG. 9A, a culture medium for growth of an interface neuron 960 may be disposed in the recess 920. Furthermore, the neural probe may include a membrane covering the opening of the recess 920.

When the neural probe is implanted in the body, neurites extending from the interface neuron 960 may be synaptically connected to a neuron C in the body. The intracellular recording interface 930 may measure a nerve signal of the neuron C in the body may be measured by measuring the intracellular potential of the interface neuron 960. The intracellular potential measured by the intracellular recording interface 930 may be transmitted to an external device through the signal transmission path 940.

As illustrated in FIG. 9A, in an embodiment, one interface neuron 960 may correspond to one intracellular recording interface 930. Alternatively, as illustrated in FIG. 9B, in another embodiment, a plurality of intracellular recording interfaces 931 to 933 and a plurality of interface neurons 961 to 963 may be disposed in the recess 920. In another embodiment, one interface neuron 962 may correspond to the intracellular recording interfaces 931 to 933. When the intracellular potential of one interface neuron 962 is measured using the intracellular recording interfaces 931 to 933, a highly reliable measurement value may be obtained.

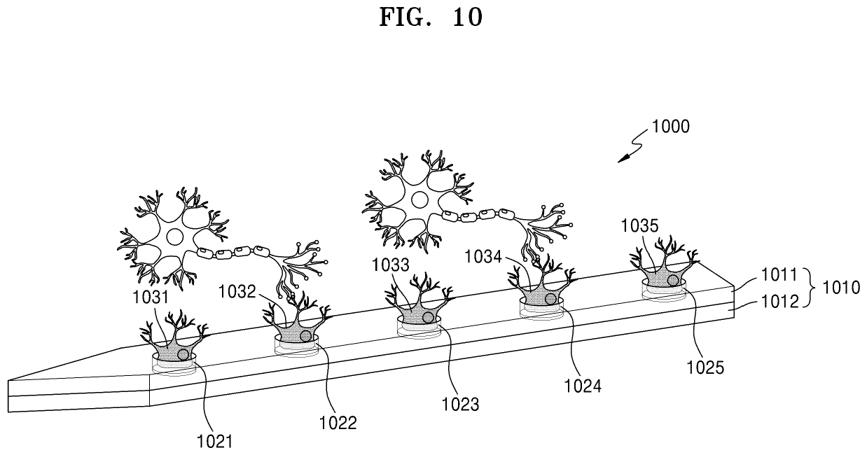

FIG. 10 is a view for describing a neural probe 1000 according to an embodiment.

The descriptions according to some embodiments of the disclosure may be applied to the neural probe 1000 of FIG. 10. Redundant descriptions thereof are omitted.

The neural probe 1000 may be implanted in the body to interface with neurons. In an embodiment, the neural probe 1000 may measure the intracellular Ca²⁺ concentration of neurons.

The neural probe 1000 may include a main body 1010, recesses 1021 to 1025, and optical interfaces disposed in the recesses 1021 to 1025.

An optical interface configured to measure the intracellular Ca²⁺ concentration may be disposed in each of the recesses 1021 to 1025. Interface neurons 1031 to 1035 may be disposed in the recesses 1021 to 1025. The optical interface may be configured to measure the intracellular Ca²⁺ concentration of each of the interface neurons 1031 to 1035.

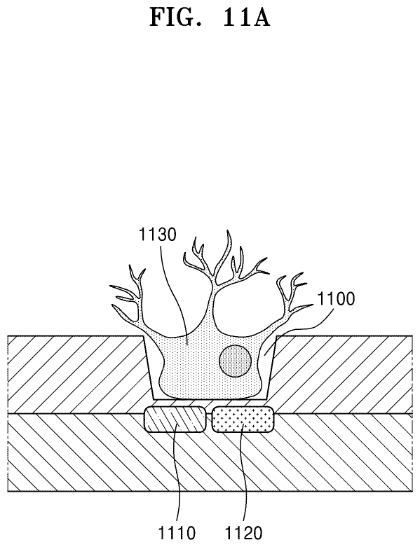

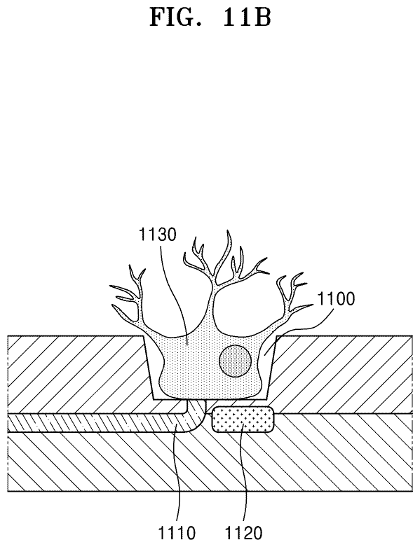

FIGS. 11A and 11B are enlarged views of a recess 1100 of a neural probe according to some embodiments.

The descriptions according to some embodiments of the disclosure may be applied to the neural probe of FIGS. 11A and 11B. Redundant descriptions thereof are omitted.

Fura-2 may be introduced into an interface neuron 1130. Fura-2 is a fluorescent dye that sensitively changes according to a calcium concentration change. Fura-2 may be characteristically excited to light having a wavelength of about 380 nm when the intracellular calcium concentration is relatively low, and to light having a wavelength of about 340 nm when the intracellular calcium concentration is relatively high. Accordingly, the intracellular calcium concentration may be measured based on a ratio of fluorescence intensities in wavelengths of 340 nm and 380 nm.

The neural probe according to an embodiment may include an optical interface. The optical interface may include an optical transmitter 1110 and an optical receiver 1120. For example, the optical transmitter 1110 may be a micro-light-emitting diode (micro-LED) as illustrated in FIG. 11A or an optical fiber as illustrated in FIG. 11B, but the disclosure is not limited thereto. For example, the optical receiver 1120 may be a photodiode, but the disclosure is not limited thereto.

The optical transmitter 1110 and the optical receiver 1120 may be disposed on a bottom surface of the recess 1100. The optical transmitter 1110 may emit light of a wavelength of about 340 nm or 380 nm to a neuron. The optical receiver 1120 may convert fluorescence intensity of the light of a wavelength of about 340 nm and 380 nm into an electrical signal.

The electrical signal measured by the optical receiver 1120 may be transmitted to an external device, the intracellular calcium concentration of the interface neuron 1130 may be measured based on a ratio of fluorescence intensity.

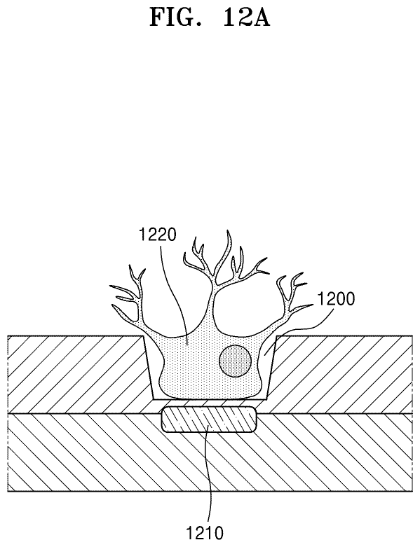

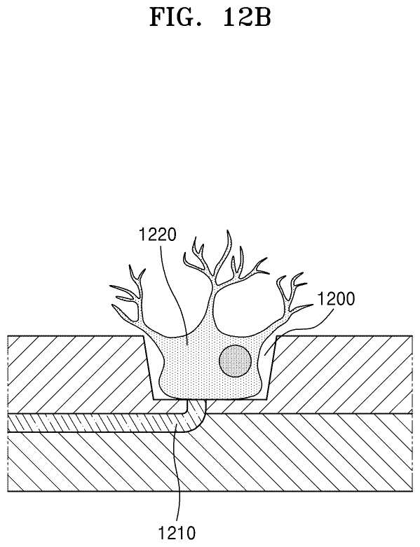

FIGS. 12A and 12B are enlarged views of a recess 1200 of a neural probe according to some embodiments.

The descriptions according to some embodiments of the disclosure may be applied to the neural probe of FIGS. 12A and 12B. Redundant descriptions thereof are omitted.

An optogenetic actuator may be introduced into an interface neuron 1220. The optogenetic actuator is protein that controls an action of a neuron when light energy of a specific wavelength is transmitted to cells. For example, the optogenetic actuator may include at least one of channelrhodopsin, halorhodopsin, or archaerhodopsin, but the disclosure is not limited thereto.

The neural probe according to an embodiment may include an optical interface 1210. For example, the optical interface 1210 may be a micro-LED as illustrated in FIG. 12A or an optical fiber as illustrated in FIG. 12B, but the disclosure is not limited thereto. The optical interface 1210 may be disposed on a bottom surface of the recess 1200.

The optical interface 1210 may emit light of a specific wavelength to the interface neuron 1220. The interface neuron 1220 may be stimulated or suppressed by light, and other neurons in the body connected in a chain to the interface neuron 1220 may be stimulated or suppressed. By introducing the optogenetic actuator into the interface neuron 1220, the action of neurons in the body connected to the interface neuron 1220 may be delicately controlled.

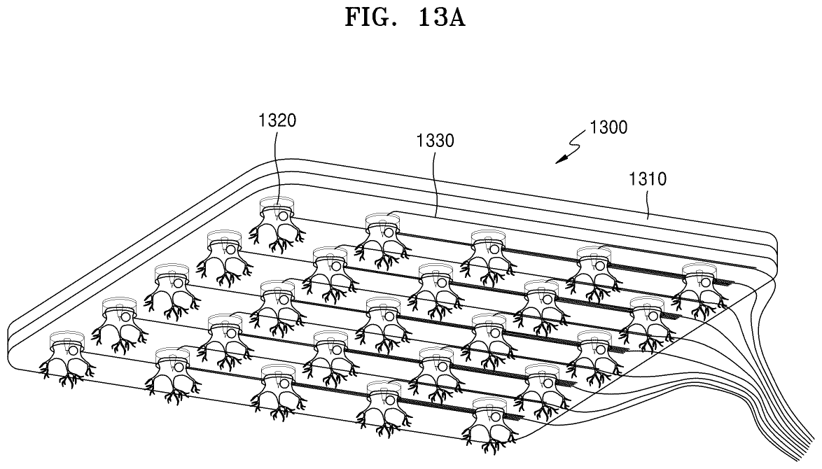

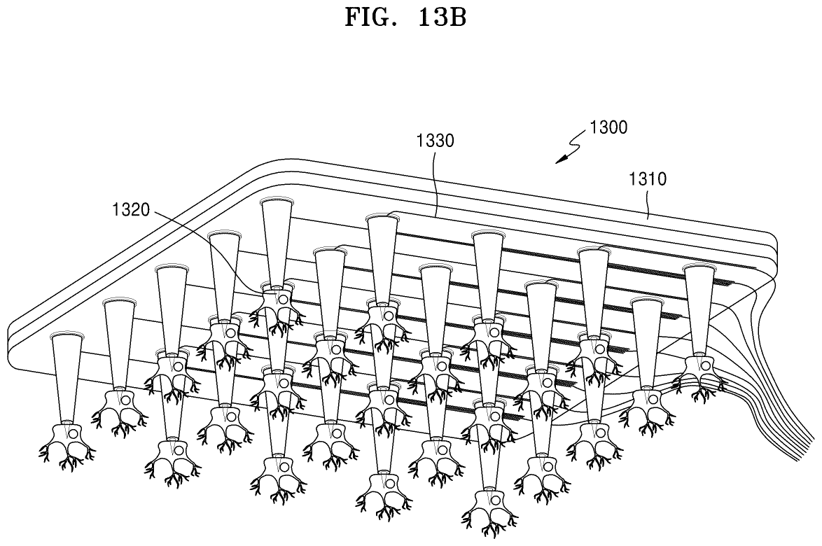

FIGS. 13A and 13B are views for describing a neural chip 1300 according to some embodiments.

The description of the neural probe according to some embodiments of the disclosure may be applied to the neural chip 1300 of FIGS. 13A and 13B. Redundant descriptions thereof are omitted.

The neural chip 1300 may be attached to a brain surface or cortex and interface with neurons in the brain. In an embodiment, the neural chip 1300 may measure nerve signals (i.e., electrical signals) of the neurons in the brain. In an embodiment, the neural chip 1300 may transmit electrical signals to the neurons in the brain.

The neural chip 1300 may include a main body 1310, a protruding electrode array 1320, and signal transmission paths 1330.

As illustrated in FIG. 13A, the protruding electrode array 1320 may be disposed on a surface of the main body 1310. Each protruding electrode may extend outward from the surface of the main body 1310. Alternatively, as illustrated in FIG. 13B, the main body 1310 may have a Utah array structure, and the protruding electrode array 1320 may be disposed on the tips of a Utah array.

The protruding electrode array 1320 is an intracellular recording interface configured to measure intracellular potentials, and an interface neuron may be disposed in each protruding electrode. Each protruding electrode may measure the intracellular potential of an interface neuron that is synaptically connected to a neuron in the brain. The intracellular potentials measured by the protruding electrode array 1320 may be transmitted to an external device through the signal transmission paths 1330.

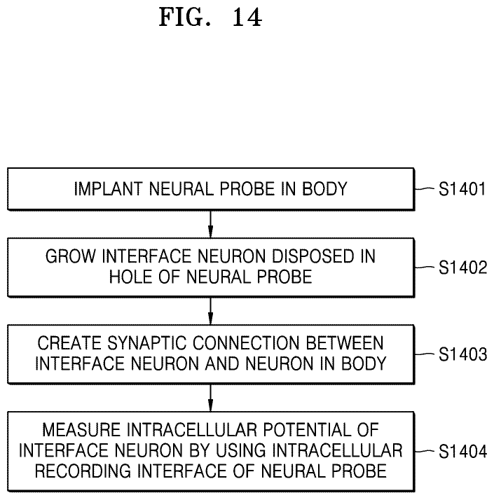

FIG. 14 is a flowchart of a method of interfacing a neural probe with neurons in vivo, according to an embodiment.

In operation S1401, a neural probe is implanted in the body.

In an embodiment, an interface neuron may be cultured in each hole of the neural probe in vitro. For example, interface neurons may be cultured for about 5 to 7 days, but the disclosure is not limited thereto. The neural probe with cultured interface neurons may be implanted in the body.

In operation S1402, the interface neuron disposed in each hole of the neural probe may grow.

In an embodiment, the growth of the interface neuron may be promoted by a chemical substance included in the hole. Alternatively, the growth of the interface neuron may be promoted by an electrical stimulus applied by an intracellular recording interface.

In operation S1403, a synaptic connection may be created between the interface neuron and a neuron in the body.

In an embodiment, the interface neuron may be synaptically connected to the nearest neuron in the body. The interface neuron and a neuron in the body may be connected by an inhibitory synapse. Alternatively, the interface neuron and a neuron in the body may be connected by an excitatory synapse. Alternatively, the interface neuron and a neuron in the body may be connected by a dendrodendritic synapse.

In an embodiment, the interface neuron may be synaptically connected to a plurality of nearest neurons in the body. The interface neuron and a plurality of neurons in the body may be connected by inhibitory synapses or excitatory synapses.

In operation S1404, the intracellular potential of an interface neuron may be measured by using the intracellular recording interface of the neural probe.

In an embodiment, as a neuron in the body emits inhibitory neurotransmitters, the interface neuron may become hyperpolarized. In this case, the subthreshold oscillations of the interface neuron may be measured by the intracellular recording interface. Alternatively, as a neuron in the body emits excitatory neurotransmitters, the interface neuron may fire. In this case, the action potential of the interface neuron may be measured by the intracellular recording interface.

In an embodiment, the neural probe may measure the nerve signals of the neurons in the body by measuring the intracellular potential of the interface neurons using the intracellular recording interface.

The disclosure may provide a neural probe capable of measuring subthreshold oscillations and an action potential by measuring an intracellular potential.

Furthermore, the disclosure may provide a neural probe capable of measuring a nerve signal with a high SNR by measuring the intracellular potential of an interface neuron creating a direct synaptic connection to a neuron in the body.

Furthermore, the disclosure may provide a neural probe capable of measuring a nerve signal with a high yield by using a plurality of interface neurons that are uniformly arranged.

Furthermore, the disclosure may provide a neural probe capable of measuring a nerve signal of a neuron in the body that is located relatively far away by creating a synaptic connection to the neuron in the body that is located relatively far away as the interface neuron extends a neurite.

Furthermore, the disclosure may provide a neural probe capable of measuring the intracellular potential of one interface neuron by arranging one interface neuron in one intracellular recording interface.

Furthermore, the disclosure may provide a neural probe adaptive to an environment in the body by growing an interface neuron according to the environment in the body.

Furthermore, according to the disclosure, the growth of an interface neuron may be promoted by applying an electrical stimulus to the interface neuron.

Furthermore, according to the disclosure, a neuron in the body connected to an interface neuron may be stimulated by applying an electrical stimulus or an optical stimulus to the interface neuron.

The effects achieved from the embodiments of the disclosure are not limited to the effects described above, and other various effects that are not mentioned herein would be clearly understood by a person skilled in the art from the description of the present invention. In other words, unintended effects by working the embodiments of the present disclosure can also be derived by one of skilled in the art from the embodiments of the disclosure.

It should be understood that embodiments described herein should be considered in a descriptive sense only and not for purposes of limitation. Descriptions of features or aspects within each embodiment should typically be considered as available for other similar features or aspects in other embodiments. For example, each constituent element described to be a single type may be embodied in a distributive manner. Likewise, the constituent elements described to be distributed may be embodied in a combined form.

While one or more embodiments have been described with reference to the figures, it will be understood by those of ordinary skill in the art that various changes in form and details may be made therein without departing from the spirit and scope of the disclosure as defined by the following claims.

What is claimed is:

1. A neural probe for interfacing with neurons in vivo, the neural probe comprising:
a main body;
a hole comprising a first opening formed in an upper surface of the main body and a second opening formed in a lower surface of the main body; and
an intracellular recording interface disposed in the hole and configured to measure an intracellular potential.

2. The neural probe of claim 1, further comprising an interface neuron disposed in the hole.

3. The neural probe of claim 2, wherein the interface neuron is synaptically connected in vivo to a neuron in a body.

4. The neural probe of claim 2, further comprising a culture medium disposed in the hole to allow the interface neuron to grow beyond at least one of the first opening and the second opening.

5. The neural probe of claim 2, further comprising a chemical substance disposed in the hole to promote growth of the interface neuron.

6. The neural probe of claim 2, wherein the intracellular recording interface is configured to measure an intracellular potential of the interface neuron.

7. The neural probe of claim 1, wherein the intracellular recording interface is located at a same distance from the first opening and the second opening.

8. The neural probe of claim 1, further comprising an interface neuron disposed in the hole,
wherein the intracellular recording interface comprises a protruding electrode configured to measure the intracellular potential of the interface neuron.

9. The neural probe of claim 8, wherein the protruding electrode extends from an inner surface of the hole to an inner space of the hole.

10. The neural probe of claim 8, wherein the protruding electrode extends in a direction intersecting a height direction of the main body.

11. The neural probe of claim 8,
wherein the protruding electrode penetrates a cell membrane of the interface neuron.

12. The neural probe of claim 8, further comprising an insulating film covering a portion of the protruding electrode exposed to outside of the interface neuron.

13. The neural probe of claim 8, wherein, the protruding electrode is configured to apply an electrical stimulus to the interface neuron to promote growth of the interface neuron.

14. The neural probe of claim 1, further comprising an interface neuron disposed in the hole,
wherein the intracellular recording interface comprises a planar patch clamp configured to measure an intracellular potential of the interface neuron.

15. The neural probe of claim 1, wherein the first opening and the second opening have a same size.

16. The neural probe of claim 1, wherein the main body comprises an upper main body in which the first opening is formed and a lower main body in which the second opening is formed.

17. The neural probe of claim 1, further comprising a signal transmission path through which an intracellular potential measured by the intracellular recording interface is transmitted to outside.

18. The neural probe of claim 1, further comprising a first membrane covering the first opening.

19. The neural probe of claim 1, further comprising a second membrane covering the second opening.

20. A method of interfacing a neural probe with neurons in vivo, the method comprising:
implanting the neural probe in a body;
growing an interface neuron disposed in a hole of a neural probe;
creating a synaptic connection between the interface neuron and a neuron in the body; and
measuring an intracellular potential of the interface neuron by using an intracellular recording interface of the neural probe.
